(19) United States
(12) Patent Application Publication
(10) Pub. No.: US 2024/0337631 A1
(43) Pub. Date: Oct. 10, 2024

(54) TITRATION-BASED ANALYTICAL METHOD FOR MEASUREMENT OF AU IN AU-SULFITE PROCESSING SOLUTION

(71) Applicant: ECI TECHNOLOGY, INC., Totowa, NJ (US)

(72) Inventor: Vishal Parekh, Parsippany, NJ (US)

(73) Assignee: ECI TECHNOLOGY, INC., Totowa, NJ (US)

(21) Appl. No.: 18/297,080

(22) Filed: Apr. 7, 2023

(51) Int. Cl. G01N 31/16; C25D 3/02

(52) U.S. Cl. CPC G01N 31/164; C25D 3/02

(57) ABSTRACT

The disclosed subject matter relates to techniques for a titration-based analytical method for measuring gold (Au) concentration in Au processing solution. An example method can include an addition of a complexing agent and/or pH adjustment to achieve a sharp inflection point in titration curve.

### BACKGROUND

The disclosed subject matter relates to analytical chemistry, and more specifically, to a titration-based analytical method for the measurement of Au in Au processing solutions.

Various methods can be used to analyze gold and concentration thereof in a solution. Certain methods have been applied to processing solutions. Processing solutions containing gold in the form of a soluble sulfite complex can avoid more dangerous gold cyanide complexes. Gold sulfite baths can provide improved ductility and throwing power, good alloy deposition, and tolerance to impurities.

Certain analytical techniques have been used to determine gold concentration in a solution, including Atomic Absorption Spectroscopy (AAS), Inductively Coupled Plasma (ICP) and Polarography, XRF and Spectroscopy after reacting with a selective reagent. Unfortunately, each of these methods can suffer from disadvantages such as open flame, complicated instrumentation, and/or use of toxic reagents. These methods can also suffer from difficulties in automation, and can yield inaccurate results. Furthermore, the use of toxic reagents can render them unsuitable for use in a cleanroom environment of a semiconductor fab.

As a result, there is a need for a simple and reliable method to analyze the concentration of gold in gold-sulfite electroplating solutions, which can be easily automated and does not require the use of toxic reagents or complicated instrumentation.

### SUMMARY

To solve the problem of inaccuracy and toxicity in the measurement, the disclosed subject matter provides simple and reliable analytical methods for the quantitative measurement of gold (Au) concentration in Au-sulfite electrodeposition solution.

The disclosed matter provides titration-based analytical methods for measuring Gold (Au) in Au-sulfite processing solutions. An example method includes adding a complexing agent having a predetermined concentration to the processing solution; adding Au-sulfite solution to the processing solution; adding a metallic salt having a predetermined concentration to the processing solutions; and determining the concentration of Au by measuring the endpoint of a back titration. The complexing agent reacts with Au in a first reaction, and the metallic salt reacts with the remaining complexing agent in a second reaction.

In certain embodiments, the first product of the first reaction has a larger stability constant than Au-sulfite.

In certain embodiments, the second product of the second reaction has a less stability constant than the first product of the first reaction.

In certain embodiments, the first reaction generates a first precipitation product, and the second reaction generates a second precipitation product.

In certain embodiments, the processing solution is an electrodeposition solution.

In certain embodiments, the processing solution includes a plating metal selected from nickel, cobalt, iron, and combinations thereof.

In certain embodiments, the complexing agent is selected from Thiourea, EDTA, Thiosulfate, nitrilotriacetic acid, iminodisuccinic acid, polyaspartic acid, S,S-ethylenediamine-N,N-disuccinic acid, methylglycinediacetic acid, L-Glutamic acid, N,N-diacetic acid, salts thereof, and combinations thereof.

In certain embodiments, the method further includes an addition of a pH adjustment reagent to the processing solution.

In certain embodiments, the method further includes an addition of a pH adjustment reagent to the processing solution before adding Au-sulfite solution.

In certain embodiments, the pH adjustment reagent decreases pH values of the processing solution.

In certain embodiments, the pH adjustment reagent is selected from nitric acid, hydrochloric acid, sulfuric acid, and combinations thereof.

In certain embodiments, the metallic salt is selected from Ag, Cu, Fe, Al, and combination thereof.

In certain embodiments, the back titration includes determining the Au concentration based on a difference between an amount of endpoint volume from a blank analysis and endpoint of an analyte analysis.

In certain embodiments, the method further includes tracking the titration using an Ag-billet electrode.

### BRIEF DESCRIPTION OF THE DRAWINGS

Reference will now be made in detail to the various exemplary embodiments of the disclosed subject matter, which are illustrated in the accompanying drawings. The accompanying drawings, where like reference numerals refer to identical or functionally similar elements throughout the separate views, serve to further illustrate various embodiments and to explain various principles and advantages all in accordance with the disclosed subject matter.

FIG. 1 provides an image showing expected and measured Au concentration in an embodiment of the disclosed subject matter.

FIG. 2A provides results of titrations with different expected concentrations in the potential versus the titrant volume in an embodiment of the disclosed subject matter. FIG. 2B provides results of titrations with different expected concentrations in the slope versus the titrant volume in an embodiment of the disclosed subject matter.

FIG. 3 provides results of titrations data points at an expected concentration in an embodiment of the disclosed subject matter.

FIG. 4A provides results of titrations with three complexing agents in the potential versus the titrant volume in an embodiment of the disclosed subject matter. FIG. 4B provides results of titrations with three complexing agents in the slope versus the titrant volume in an embodiment of the disclosed subject matter.

It is to be understood that both the foregoing general description and the following detailed description are exemplary and are intended to provide further explanation of the disclosed subject matter.

### DETAILED DESCRIPTION

The disclosed subject matter provides methods of using titration to measure the amount of Au in a solution containing Au analyte. Such methods can be applied into Au solution for different purposes, like analyzing, monitoring, measuring, or determining Au concentration in Au solution.

For clarity, but not by way of limitation, the detailed description of the presently disclosed subject matter is divided into the following subsections:
I. Definitions
Methods of Titration II.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosed subject matter and how to make and use them.

For purposes of interpreting this specification, the following definitions will apply and whenever appropriate, terms used in the singular will also include the plural and vice versa.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, e.g., with respect to biological systems or processes, the term can mean within an order of magnitude, within 5-fold, and within 2-fold, of a value.

As used herein, the term “high level” when used in context of a concentration of a metal ion in a solution refers to a concentration in the range of grams per liter (g/L).

As used herein, the term “low level” when used in context of a concentration of a metal ion in a solution refers to a concentration in the range of parts per million (ppm).

As used herein, the term “trace” refers to a concentration less than 1000 ppm. In certain embodiments, trace levels refer to a concentration range of from 0.1 ppm to 1000 ppm.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. Ranges disclosed herein, for example, “between about X and about Y” are, unless specified otherwise, inclusive of range limits about X and about Y as well as X and Y. With respect to sub-ranges, “nested sub-ranges” that extend from either endpoint of the range are specifically contemplated. For example, a nested sub-range of an exemplary range of 1 to 50 can include 1 to 10, 1 to 20, 1 to 30, and 1 to 40 in one direction, or 50 to 40, 50 to 30, 50 to 20, and 50 to 10 in the other direction.

As used herein, the term “predetermined concentration” as used herein refers to a known, target, standard or optimum concentration of a component in a solution.

As used herein, the term “selective” or “selectively” refers to, for example, the particular monitoring, measurement, or determination of a characteristic of a specific or particular component. For example, the selective measurement of an ion refers to the measurement of one particular or predetermined target ion from a plurality of the ions present in solution.

As used herein, the term “accurate” or “accurately” refers to, for example, a measurement or determination that is relatively close to or near an existing or true value, standard, or known measurement or value. In certain embodiments, the measurement or determination accuracy error is less than ±5%, having a standard deviation less than 0.02, and/or a residual standard deviation (RSD) less than 4%.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

As used herein, the term “processing solution” refers a chemical solution which is used to analyze the concentration of a substance in the solution by reacting it with a known amount of a standard solution. Processing solutions are used in several industries, including electro/electroless plating, metallurgical, chemical, pharmaceutical, and other industries in which measuring, monitoring and control of an analyte is needed.

As used herein, the term “stability constant”, also known as formation constant K, refers to a measure of the strength of the interaction between two or more chemical species, such as a metal ion and a ligand. It quantitatively describes the equilibrium constant of a complex formation reaction in a solution, which refers to the degree of stability of the resulting complex. The larger the stability constant, the more stable the complex is, and the greater/stronger the tendency of the reactants to form the complex.

As used herein, the term “complexing Agent” refers a substance that forms a complex with a metal ion, to control the concentration of metal ions in a solution for purpose, e.g., a titration. The use of complexing agents can have several important roles on titration chemistry. For example, but no ways of limitation, the addition of a complexing agent can shift the equilibrium towards the formation of a metal-ligand complex, which can make it easier to measure the concentration of the metal ion. Certain complexing agents can include but on ways of limitations, EDTA, cyanide, dithizone, thiourea, thiosulfate, and NTA.

As used herein, the term “Ag-billet electrode” refers to a type of electrode used in electrochemistry to measure the concentration of certain ions in a solution. It can include a silver rod that is partially immersed in the solution being analyzed. When an electric potential is applied to the Ag-billet electrode, silver ions are released into the solution, which react with other ions in the solution to produce an electrical current. By measuring the current and/or potential, the concentration of the ions being measured can be determined.

As used herein, the term “back titration”, also known as reverse titration, refers to a type of titration method that is used to determine or measure the amount of a substance present in a sample. It can be used when direct titration is not possible due to the properties of the sample, or the nature of the reaction being investigated.

As used herein, the term “blank analysis”, also known as “blank titration”, refers to a control titration process that is carried out in the absence of the analyte (the substance being analyzed) in order to correct for any impurities or contaminants in the titrant or the solvent used in the titration. A blank analysis can be carried out by using the same amount of solvent and the same amount of titrant as in the actual titration, but without adding any analyte. The endpoint of the blank analysis is determined using the same method as for the actual titration (such as using an indicator or an instrument like a potentiometer or pH meter). The volume of titrant used in the blank analysis (also known as blank vend) is then subtracted from the volume of titrant used in the actual titration to determine the volume of titrant required to neutralize the analyte in the sample and correct for any impurities or contaminants that may have affected the titration. The use of a blank analysis can be important to ensure that the titrant and solvent are free from interfering substances that can affect the accuracy of the titration.

As used herein, the term “analyte analysis”, also known as analyte titration, refers to a control titration process that is carried out in the presence of the analyte (the substance being analyzed) in order to measure the endpoint of titrant in the solution including analyte, which is used to compare to the endpoint of titrant in the solution in the blank titration for determining the analyte concentration.

As used herein, the term “titrant” refers to a standard solution comprising a known concentration of a reagent that chemically reacts with a “reactant” or “unknown species” whose concentration in a sample solution is to be determined. A “titration” is an analytical procedure involving repeated standard addition of a known volume of a titrant solution to an analysis solution (comprising the sample solution), coupled with monitoring the concentration of an indicator species, which participates in the reaction between the titrant and the reactant, or is indirectly affected by this reaction.

As used herein, the term “equivalence point” refers to the point in a titration at which the reaction between the titrant and the reactant is complete, corresponding to a stoichiometric balance between the number of moles of the titrant and the number of moles of the reactant with respect to formation of a compound or complex.

As used herein, the term “titration endpoint” refers to a relatively rapid change in the concentration of the indicator species as additional titrant is added to the analysis solution after the equivalence point has been reached. The concentration of the unknown species in the sample solution can be calculated from the volume of titrant solution added to the analysis solution at the equivalence point (approximately equivalent to the endpoint). A “back titration” involves standard addition (to an analysis solution) of a back-titration reagent that reacts with the unknown species. An excess of the back-titration reagent is added to the analysis solution and then reacted with a titrant in a titration back to the equivalence point.

As used herein, the term “titration curve” refers to a plot of the concentration of a titration indicator species in an analysis solution, or a parameter proportional to this concentration, as a function of the volume of titrant solution added to the analysis solution. It can be more convenient to utilize a concentration parameter that is proportional to the concentration of the indicator species, especially when the indicator species participates in a complexation reaction involving competing complexing agents. The endpoint for the titration can be determined from a curve feature corresponding to a rapid change in the concentration of the indicator species, such as a curve knee or inflection point. Detection of the titration endpoint can be facilitated by differentiating the titration curve, which converts an inflection point into a peak. Titration data can be handled as titration curves or plots but such data can be tabulated and used directly, e.g., by a computer, and the term “titration curve” includes tabulated data.

The methods of the disclosed subject matter can be applied to various types of solutions including electroplating solution. In certain embodiments, the processing solution can include Au-sulfite. A person skilled in the art will appreciate that a wide variation of Au-sulfite or forms of Au and its compounds are suitable for use herein. In certain embodiments, the processing solution can include Au-sulfite.

### II. Methods for Titration

The disclosed subject matter provides techniques for analysis and measurement of Au in Au-sulfite processing solutions such as electrodeposition solutions. In certain aspects, techniques of the disclosed subject matter can provide a safe and toxic-free method with accurate, rapid, and efficient measurement results for Au concentration analysis in processing solutions including Au-sulfite. In certain embodiments, methods of the disclosed subject matter can aid in process control of Au in metal alloy plating baths.

Such methods use much simpler titration method with non-toxic reagents or complicated analytical instrumentation. In addition, such methods are faster, less cost and easily automated in cleanroom environment of manufacture industry, e.g., semiconductive industry.

The methods of the disclosed subject matter can be incorporated in existing as an extension of application, including determining, monitoring and/or analyzing the Au(I) concentration in Au-sulfite electrodeposition solution. Further, such methods of the disclosed subject matter can be applied to a new chemical monitoring and/or measuring system to analyze Au(I) concentration in Au-sulfite electrodeposition solution.

In an exemplary method, a titration-based analytical method for measuring Gold (Au) in Au-sulfite processing solutions can be implemented as follows. A complexing agent having a predetermined concentration to the processing solution is added thereto. An Au-sulfite solution is added to the processing solution, followed by the addition of a metallic salt having a predetermined concentration. The concentration of Au is determined by measuring the endpoint of a back titration. In such a method, the complexing agent reacts with the complexing agent in a first reaction, and the metallic salt reacts with the remaining complexing agent in a second reaction.

In the first reaction during the titration-based analytical method for measurement for Au—Au-sulfite, the Au-sulfite compound is broken after adding a complexing agent, which can be achieved via forming a new Au complex (Au(I)—X, where X is a ligand of the complexing agent), with a larger stability constant, (e.g., thereof K≥10²³, much more Au-sulfite, K˜10¹⁰) in the processing solution. In a selective way of implementation, substantially all of Au can be precipitated out from the processing solution as a precipitant.

After all the Au(I) has been substantially removed out in the processing solution, the second complex can be produced via the second reaction between the excess/remaining complexing agent with a metal salt, which can be achieved via maintaining the second complex (M-X, M is a metal element and X is a ligand of the complexing agent) with less stability constant, e.g., thereof 10¹⁰≤K≤10²³. Accordingly, the second complex can be precipitated out during the second reaction. The endpoint of titration can be measured at the stop points of the second reaction between remaining complexing agent and the metal salt. Combined with a back titration, the titration volume/concentration of the first reaction can be determined based on the difference between titration volume/concentration of the back titration and the titration volume/concentration of the second reaction.

A complexing agent X of predetermined concentration can be added to form new Au complex. As embodied herein, the first reaction can be represented by the following chemical equation:

$$[ W ] Au - su lfite + [ S 1 ] X -- > {Au}_{y 1} - {X}_{x 1} \qquad ( 1 )$$

y1 and x1 are standard stoichiometric ratio of Au-sulfite and complexing agent X, respectively; W and S1 are the amounts of reactants thereof in the first reaction, respectively.

A metallic salt can be added as titration (Me⁺, metal element) of predetermined concentration to the processing solution to cause a reaction between at least a portion of the metallic salt (Me⁺) and substantially all of the precipitating agent (X⁻) remaining in the processing solution that did not react with the Au(I). As embodied here, the second reaction can be represented by the following equation:

$$[ T 2 ] {Me}^{+} + [ S 2 ] {X}^{-} -> {Me}_{y 2} - {X}_{x 2} \qquad ( 2 )$$

where y2 and x2 are standard stoichiometric ratio of metal salt and complexing agent X, respectively; T2 and S2 are the amounts of reactants in the second reaction, respectively. Notably, T2 can be readily determined by the endpoint of (2) via titration, and accordingly, S2 can be calculated via stoichiometric ratio.

An endpoint of a back titration can be measured using the same metallic salt (Me⁺) as titrate. In a blank analysis, the reaction can be represented by the following equation:

$$[ T ] {Me}^{+} + [ S ] {X}^{-} -> {Me}_{y 2} - {X}_{x 2} \qquad ( 3 )$$

where T and S are the amounts of reactants in the blank analysis without analyte solution, respectively. Notably, T can be readily determined by the endpoint of (3) via titration, and S is a predetermined value.

The titration volume associated with the endpoint in (2) can be subtracted from the back titration volume associate with the endpoint of (3). To obtain the difference, the volume of the complexing agent reacting with Au in (1) can be calculated, and consequently the concentration of Au(I) in Au-Sulfite processing solution can be determined based on stoichiometric ratio between the reactants in the equations.

As embodied herein, the Au concentration can be calculated as follows:

$$S = T * y 2 / x 2 \qquad ( 4 )$$

$$S 2 = T 2 * y 2 / x 2 \qquad ( 5 )$$

$$S 1 = S - S 2 \qquad ( 6 )$$

$$W = S 1 * x 1 / y 1 \qquad ( 7 )$$

$${Concentration}_{Au} = W / V \qquad ( 8 )$$

$$N \text{ote:} \\ V s a predetermined volume of analyte solution adding into the processing solution .$$

In certain embodiments, a back titration can comprise a blank analysis and an analyte analysis. In certain embodiments, the analyte titration can include adding Au-sulfite into the processing solution as an analyte solution. The back titration can determine Au concentration based on the difference between an amount of endpoint from a blank titration and endpoint of an analyte titration. In certain embodiments, the method includes tracking the titration by using a Ag-billed electrode.

In certain embodiments, Au-sulfite can react with the complexing agent, and the metallic salt can react with the remaining complexing agent. The complexing agent in the processing solution is generally used as a metal chelator to bond metal ions, typically the ions being analyzed or measured. In certain embodiments, the first reaction between Au-sulfite and the complexing agent and the second reaction between metallic salt and the remaining complexing agent can both produce precipitations for determining titration endpoint.

To implement the above titration with expected results, a selection of the complex agents and metal salts can be desirable to cause precipitations in the solutions. Generally, in a solution, the larger stability constant K of a compound is, the greater the tendency of forming the compound (e.g., precipitating out in the solution) is. Therefore, the disclosed subject matter provides a complexing agent which can enable a formation of the product (Au—X) in the first reaction having a larger K than Au-sulfite. Also, a formation of the product (Me-X) in the second reaction having a larger K than Au-sulfite but less than Au—X. This can implement the titration-based analytical method for Au in Au-sulfite processing solution.

In certain embodiments, the complexing agent can be one or more of Thiourea, EDTA, Thiosulfate, nitrilotriacetic acid, iminodisuccinic acid, polyaspartic acid, S,S-ethylenediamine-N,N′-disuccinic acid, methylglycinediacetic acid, L-Glutamic acid, N,N-diacetic acid, salts thereof, and combinations thereof. A person of ordinary skill in the art would readily appreciate the complexing agent can include standard derivation, e.g., chelators, chelating agers or complexers, of the above selection.

In certain embodiments, the processing solution is an electrodeposition solution. In certain embodiments, the processing solution includes a plating metal selected from nickel, cobalt, iron, aluminum and combinations thereof.

In certain embodiments, the method also includes an addition of a pH adjustment reagent to the processing solution. Au-sulfite complex is usually stable in alkaline pH (K, ˜10¹⁰) solution. When pH decreases, the complex can be broken effectively, and the Au(I) can convert to Au(III) and/or metallic Au. Such methods can break this complex and form a new Au complex (K, ≥10²³) in acidic pH solution with the addition of pH adjusting reagent. This pH adjustment reagent can develop an effective sharp inflection point for accurate quantification during titration.

In certain embodiments, the addition of a pH adjustment reagent to the processing solution can be performed selectively, e.g., before or after adding Au-sulfite solution, because the pH adjustment does not substantially affect the formation of Au(I)—X or reaction between Au and the complexing agent. In certain embodiments, the pH adjustment reagent decreases pH values of the processing solution.

In certain embodiments, the pH adjustment reagent is selected from nitric acid, hydrochloric acid, hydrochloric acid, and combinations thereof.

In certain embodiments, the metallic salt is selected from Ag, Cu, Iron, Al, and combination thereof.

In certain embodiments, the processing solution is an electrodeposition solution. In certain embodiments, the method can include an addition of a pH adjustment agent to the processing solution.

In certain embodiments, the pH adjustment agent decreases pH values of the processing solution.

Further, the methods provided by the disclosed subject matter can be utilized to some extendable application, including not limiting into, analyzing, controlling, and adjusting Au concentration in Au-sulfite processing solution.

Meanwhile, a person skilled in the art would appreciate the methods of the disclosed subject matter can be applied to similar or equivalent analyte solution, including but not limiting to, Au-sulfide, Au-sulfate, and Au-thiosulfate, upon fulfilling the above stability constant criteria.

### EXAMPLES

The disclosed subject matter will be better understood by reference to the following examples. The following examples are merely illustrative of the presently disclosed subject matter and they should not be considered as limiting the scope of the subject matter in any way. The examples utilize the following reagents and analyzers.

Reagents: Nitric acid, Thiourea, EDTA, Thiosulfate, Silver nitrate, and Au-sulfite electroplating solution.

Analyzer: ECI Qualilab EZ (for feasibility studies), CI Qualifill Libra (for final development), and Ag-billet electrode.

For the following example, these related measurement terms regarding accuracy to evaluate method can be understood and characterized as follows.

Accuracy(%)=[(Measured Average)−(Expected Value)/(Expected Value)]*100 (9)

Relative Standard Deviation, RSD(%)=(Standard Deviation*100)/(Measured Average) (10)

### Example 1: Au Concentration Determination

Methods of the disclosed subject matter provide for the measurement of Au concentrations in Au-sulfite electrodeposition solutions, with addition of pH adjustment. Au concentrations (M, g/l, expected target 0.50, low 0.25, high 1.00) in such solutions can be determined as follows.

Process I Blank analysis:

1) deionized water is added to the reaction vessel (˜50-100 ml).

2) reagent 1 (complexing agent, Thiourea) is added to the reaction vessel (˜2-5 ml depending upon analyte concentration).

3) resulting mixture is titrated using metal salt (Silver Nitrate)

3) reagent 2 (Nitric Acid) is added to the reaction vessel (˜5˜10 ml depending upon analyte concentration).

4) resulting mixture is titrated using metal salt.

5) endpoint is detected automatically via potentiometric titration, selectively either by “fixed potential” settings or “inflection point” algorithms.

6) blank vend is recorded associated with volume at the endpoint.

7) titration is stopped automatically based on “stop point” algorithm.

8) the reaction vessel and electrode were cleaned using cleaning solution (nitric acid) and deionized water.

Process II: Analyte analysis:

1) deionized water is added to the reaction vessel (˜50-100 ml).

2) reagent 1 (thiourea) is added to the reaction vessel (˜2-5 ml depending upon analyte concentration).

3) reagent 2 (nitric acid) is added to the reaction vessel (˜5˜10 ml depending upon analyte concentration).

4) analyte solution (au-sulfite) is added to the reaction vessel (˜2-6 ml depending upon analyte concentration).

5) resulting mixture is titrated using metal salt silver nitrate.

6) endpoint is detected automatically either by “fixed potential” settings or “inflection point” algorithms, the volume of titration in the second reaction is recorded.

7) analyte vend is calculated by subtracting endpoint volume from blank vend.

8) analyte concentration is calculated automatically based on vend, sample volume and titrant concentration.

9) titration is stopped automatically based on “stop point” algorithm.

10) the reaction vessel and electrode were cleaned using cleaning solution (nitric acid) and deionized water.

Table 1 summarizes the results of measurement of Au concentration based on online prototype qualification data (ECI Qualifill Libra). Calculated results for Au concentration illustrate the accuracy and precision of the method in the presently disclosed example.

TABLE 1
Measured Au, M, g/l

| Data Point | Target | Low | High |
|---|---|---|---|
| 1 | 0.502 | 0.253 | 0.999 |
| 2 | 0.471 | 0.248 | 1.015 |
| 3 | 0.492 | 0.257 | 0.991 |
| 4 | 0.498 | 0.244 | 1.004 |
| 5 | 0.475 | 0.246 | 1.018 |
| 6 | 0.472 | 0.257 | 1.014 |
| 7 | 0.494 | 0.265 | 1.016 |
| 8 | 0.496 | 0.262 | 1.005 |
| 9 | 0.479 | 0.275 | 0.995 |
| 10 | 0.492 | 0.262 | 1.004 |
| 11 | 0.480 | 0.263 | 1.009 |
| 12 | 0.473 | 0.261 | 1.023 |
| 13 | 0.493 | 0.249 | 1.007 |
| 14 | 0.490 | 0.259 | 1.009 |
| 15 | 0.517 | 0.267 | 0.952 |
| Average, g/l | 0.49 | 0.26 | 1.00 |
| Expected, g/l | 0.50 | 0.25 | 1.00 |
| Accuracy, %* | −2.36% | 3.12% | 0.39% |
| StDev | 0.01 | 0.01 | 0.02 |
| RSD, %** | 2.64% | 3.32% | 1.68% |

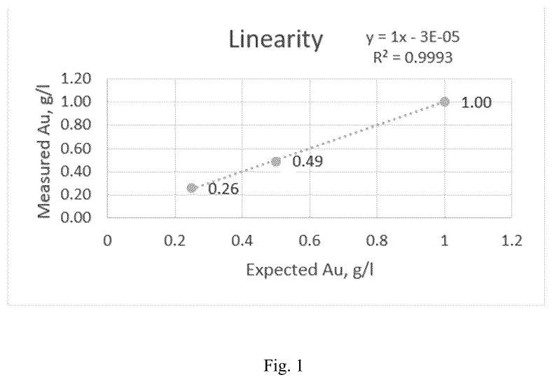

Associating with Table 1, FIG. 1 substantially shows accuracy and precision of the exemplary method in the example. The measurement method is substantially linear, as shown in FIG. 1, demonstrating that the measured values in the example increase proportionally with changes in the expected values of Au concentration in the solution.

Table 2 shows the potential curve and slope curve for titration (AgNO₃ as a titrant) in the example.

TABLE 2

| Low | | | Target | | | High | | |
|---|---|---|---|---|---|---|---|---|
| Titrant | Potential, | Slope, | Titrant | Potential, | Slope, | Titrant | Potential, | Slope, |
| Volume, ml | mV | mV/ml | Volume, ml | mV | mV/ml | Volume, ml | mV | mV/ml |
| 0 | 10.5815 | 0 | 0 | 94.889 | 0 | 0 | 190.552 | 0 |
| 0.25 | 122.782 | 67.871 | 0.207 | 104.085 | 44.425 | 0.15 | 199.972 | 62.798 |
| 0.5 | 134.562 | 47.119 | 0.414 | 113.749 | 46.686 | 0.3 | 209.167 | 61.306 |
| 0.75 | 145.121 | 42.236 | 0.621 | 124.186 | 50.421 | 0.45 | 218.933 | 65.104 |
| 1 | 155.396 | 41.097 | 0.828 | 135.234 | 53.369 | 0.6 | 230.062 | 74.192 |
| 1.25 | 166.565 | 44.678 | 1.035 | 147.4 | 58.775 | 0.75 | 243.327 | 88.433 |
| 1.5 | 179.057 | 49.967 | 1.242 | 161.784 | 69.488 | 0.9 | 259.399 | 107.151 |
| 1.75 | 193.868 | 59.245 | 1.449 | 178.507 | 80.791 | 1.05 | 279.012 | 130.751 |
| 2 | 213.175 | 77.23 | 1.656 | 200.704 | 107.23 | 1.2 | 301.412 | 149.333 |
| 2.25 | 239.258 | 104.329 | 1.863 | 228.943 | 136.42 | 1.35 | 323.98 | 145.806 |
| 2.5 | 273.01 | 135.01 | 2.07 | 255.351 | 127.575 | 1.5 | 341.98 | 124.647 |
| 2.75 | 296.285 | 93.099 | 2.277 | 261.434 | 29.387 | 1.65 | 354.228 | 81.651 |

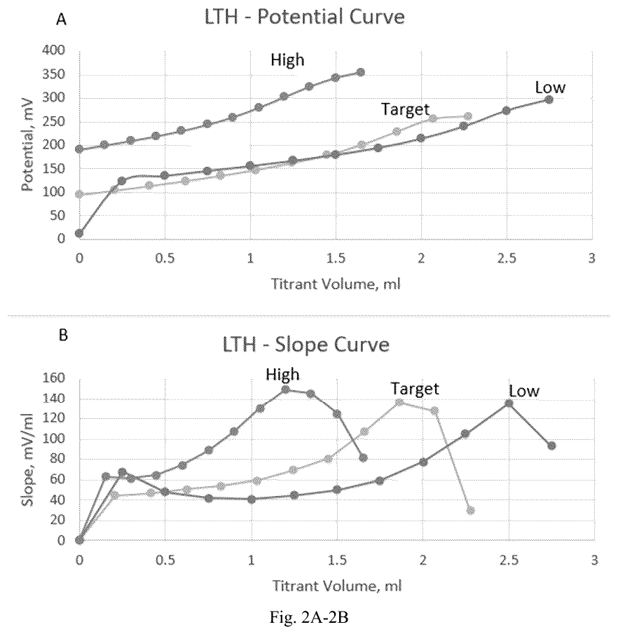

Associating with Table 2, FIGS. 2A-2B shows a potential curve and slope curve for titration herein. The endpoints for the back titration are the evident as good sharp inflection point in the curve. Remarkably, in the slope curve FIG. 2B, the inflection points and peak points for Au concentration in solutions, illustrate accuracy and precision. The Au concentration in Au-sulfite solution was calculated from the amount of analyte and complexing agent in the first reaction, via stoichiometric ratio in the first reaction. The amount of complexing agent in the first reaction was previously calculated by measuring the amount of titrant in second reaction (associated with the endpoint of analyte analysis) and the amount of titrant in blank analysis. A calculation can be referred to the above equations described.

As shown in FIGS. 2A-2B, the endpoints of the second reaction are determined by the fixed potential settings or inflection point algorithms, associating with the fixation points or inflection points in the curve. Upon the determination of endpoints, the measurement for Au concentration is calculated based on the predetermined and standard parameters via the above equations.

### Example 2: Long-Term Validation For Au Concentration Measurement

Herein, an Au-sulfite solution with an expected Au concentration at 0.50 g/l is used in the measurement method to implement the measurement. Following the same procedures and parameters as the above examples, 92 data points have been measured to test accuracy and precision of the measurement in the presently disclosed methods.

Table 3 lists data points regarding Au concentration in the example.

TABLE 3

| Data | Measured |
|---|---|
| Point | Au, g/l |
| 1 | 0.521 |
| 2 | 0.499 |
| 3 | 0.524 |
| 4 | 0.513 |
| 5 | 0.497 |
| 6 | 0.516 |
| 7 | 0.519 |
| 8 | 0.513 |
| 9 | 0.523 |
| 10 | 0.536 |
| 11 | 0.526 |
| 12 | 0.517 |
| 13 | 0.523 |
| 14 | 0.513 |
| 15 | 0.516 |
| 16 | 0.513 |
| 17 | 0.51 |
| 18 | 0.524 |
| 19 | 0.517 |
| 20 | 0.505 |
| 21 | 0.522 |
| 22 | 0.522 |
| 23 | 0.504 |
| 24 | 0.526 |
| 25 | 0.517 |
| 26 | 0.499 |
| 27 | 0.516 |
| 28 | 0.504 |
| 29 | 0.507 |
| 30 | 0.517 |
| 31 | 0.518 |
| 32 | 0.522 |
| 33 | 0.517 |
| 34 | 0.525 |
| 35 | 0.519 |
| 36 | 0.515 |
| 37 | 0.52 |
| 38 | 0.505 |
| 39 | 0.517 |
| 40 | 0.542 |
| 41 | 0.525 |
| 42 | 0.519 |
| 43 | 0.517 |
| 44 | 0.53 |
| 45 | 0.526 |
| 46 | 0.53 |
| 47 | 0.523 |
| 48 | 0.518 |
| 49 | 0.522 |
| 50 | 0.498 |
| 51 | 0.524 |
| 52 | 0.526 |
| 53 | 0.508 |
| 54 | 0.525 |
| 55 | 0.524 |
| 56 | 0.525 |
| 57 | 0.525 |
| 58 | 0.524 |
| 59 | 0.509 |
| 60 | 0.518 |
| 61 | 0.529 |
| 62 | 0.5 |
| 63 | 0.526 |
| 64 | 0.527 |
| 65 | 0.554 |
| 66 | 0.529 |
| 67 | 0.526 |
| 68 | 0.521 |
| 69 | 0.531 |
| 70 | 0.525 |
| 71 | 0.527 |
| 72 | 0.528 |
| 73 | 0.51 |
| 74 | 0.528 |
| 75 | 0.513 |
| 76 | 0.512 |
| 77 | 0.522 |
| 78 | 0.529 |
| 79 | 0.524 |
| 80 | 0.539 |
| 81 | 0.525 |
| 82 | 0.528 |
| 83 | 0.525 |
| 84 | 0.53 |
| 85 | 0.535 |
| 86 | 0.55 |
| 87 | 0.54 |
| 88 | 0.542 |
| 89 | 0.545 |
| 90 | 0.543 |
| 91 | 0.544 |
| 92 | 0.526 |
| Average, g/l | 0.52 |
| Expected, g/l | 0.50 |
| Accuracy, %* | 4.35 |
| StDev | 0.01 |
| RSD, %** | 2.15 |

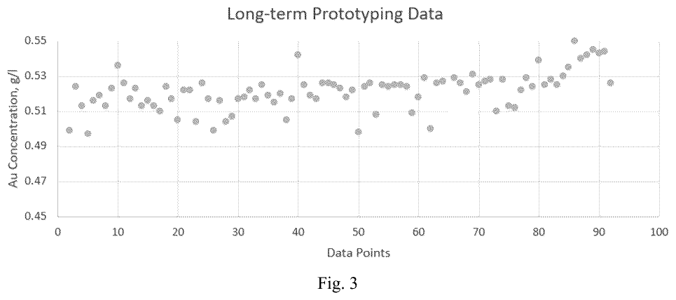

FIG. 3 shows the distribution of data points regarding Au concentration. All the data points of the example were distributed at a narrow range, about from 0.499 to 0.550, with accuracy at 4.35%, StDev at 0.01, and RSD at 2.15%. d

### Example 3: Comparison of Different Complexing Agents

In the example herein, different complexing agents were used in the method of Au concentration in solution. As described above, complexing agents with large stability constant K have a great tendency to produce stable products, e.g., as a precipitation, via complexing with Au ion in the solution. In other words, an ideal complexing agent enables to produce a complex compound (Au—X) having stability constant K substantially more than Au-sulfite (K, 10¹⁰).

Meanwhile, the metal complex (Me-X) produced in the second reaction (analyte titration) ideally has K more than Au-sulfite. Table 4 lists K of different complex Au—X and Ag—X associating with three different complexing agents in solutions. Notably, although cyanide can function as a complexing agent to complex Au in the Au-sulfite solutions, cyanide can cause significant toxic issues in the process. In addition, cyanide does not complex Ag effectively in the second reaction, according to K values on table 4.

TABLE 4

Stability Constant K (10\hat{ }x)

| Complexing Agent | Au | Ag |
|---|---|---|
| Thiourea | 23 | 13 |
| Thiosulfate | 28 | 13 |
| EDTA | Unknown | 7 |
| Cyanide | 39 | 4-8 |
| Sulfite | 10 | Unknown |

Herein, measurements with different complexing agents, Thiourea, Thiosulfate, and EDTA are implemented, using the same steps and parameters as the above examples. Table 5 lists the measurement results of data points regarding titrant volume, potential, and slope for three different complexing agents used in the Au concentration analytical method.

TABLE 5

| Thiourea | | | EDTA | | | Thiosulfate | | |
|---|---|---|---|---|---|---|---|---|
| Titrant | Potential, | Slope, | Titrant | Potential, | Slope, | Titrant | Potential, | Slope, |
| Volume, ml | mV | mV/ml | Volume, ml | mV | mV/ml | Volume, ml | mV | mV/ml |
| 0 | 94.889 | 0 | 0 | 296.834 | 0 | 0 | −66.508 | 0 |
| 0.207 | 104.085 | 44.425 | 0.5 | 307.393 | 21.118 | 0.5 | −6.897 | 119.222 |
| 0.414 | 113.749 | 46.686 | 1 | 314.88 | 14.974 | 0.65 | 3.805 | 71.343 |
| 0.621 | 124.186 | 50.421 | 1.5 | 320.74 | 11.719 | 0.807 | 12.695 | 56.706 |
| 0.828 | 135.234 | 53.369 | 2 | 325.439 | 9.399 | 1.012 | 21.973 | 45.297 |
| 1.035 | 147.4 | 58.775 | 2.5 | 329.468 | 8.057 | 1.266 | 32.125 | 39.912 |
| 1.242 | 161.784 | 69.488 | 3 | 332.906 | 6.877 | 1.549 | 42.603 | 36.961 |
| 1.449 | 178.507 | 80.791 | 3.5 | 335.958 | 6.104 | 1.854 | 53.772 | 36.735 |
| 1.656 | 200.704 | 107.23 | 4 | 338.664 | 5.412 | 2.155 | 63.619 | 32.656 |
| 1.863 | 228.943 | 136.42 | 4.5 | 341.085 | 4.842 | 2.504 | 77.738 | 40.494 |
| 2.07 | 255.351 | 127.575 | 5 | 343.323 | 4.476 | 2.761 | 89.091 | 44.169 |
| 2.277 | 261.434 | 29.387 | 5.5 | 345.398 | 4.15 | 3.011 | 100.586 | 46.011 |
| | | | 6 | 347.31 | 3.825 | 3.25 | 112.02 | 47.819 |
| | | | 6.5 | 349.08 | 3.54 | 3.48 | 123.861 | 51.401 |
| | | | 7 | 350.749 | 3.337 | 3.693 | 134.379 | 49.501 |
| | | | 7.5 | 352.295 | 3.092 | 3.919 | 145.162 | 47.536 |
| | | | 8 | 353.76 | 2.93 | 4.154 | 154.989 | 41.832 |
| | | | 8.5 | 355.123 | 2.726 | 4.427 | 159.505 | 16.586 |
| | | | 9 | 356.445 | 2.645 | | | |
| | | | 9.5 | 357.686 | 2.482 | | | |
| | | | 10 | 358.846 | 2.319 | | | |

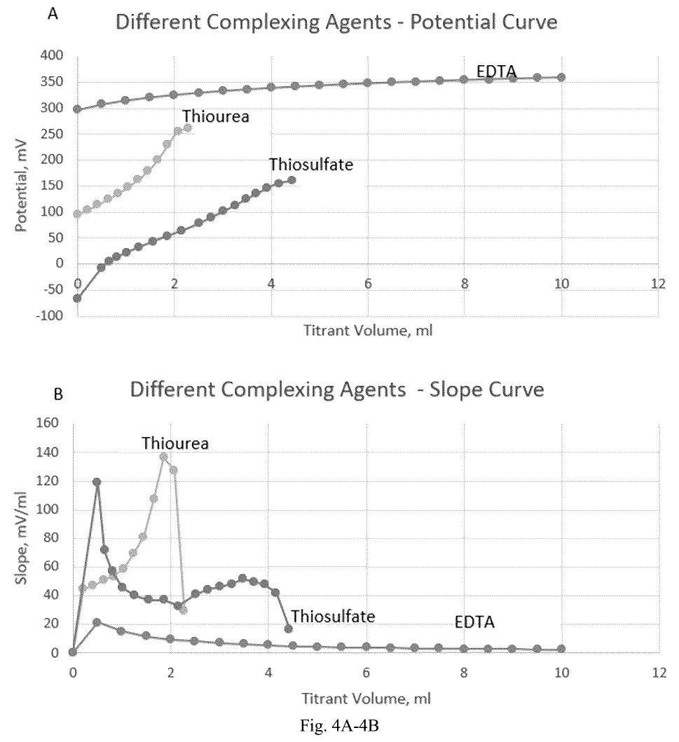

Associating with data values in Table 5, FIGS. 4A-4B shows a potential curve and slope curve in titration. In these titration curves, titration volume (AgNO₃ as titrant) is the horizontal axis. When EDTA is used as a complexing agent, the potential curve becomes a gradually flat line, and substantially the equivalent point is invisible. Therefore, EDTA is not an effective complexing agent for measuring Au concentration.

In comparison to EDTA, Thiourea or Thiosulfate is more appropriate to be used as a complexing agent. As shown in the FIGS. 4A-4B, the equivalent points of titration curve are well recognized, indicating a sharp inflection in the slope curve, as shown in FIG. 4B. Notably, when employing Thiourea as a complexing agent for the measurement of Au concentration, the slope curve is a steep as shown in FIG. 4B, almost vertical line around the equivalence point.

This is because the stability constant K of products (Au-Thiourea) in the solution is up to 10²⁸, rapidly changing K in the solution. A sudden shift from a weaker compound to a stronger compound (Au-Thiourea, having a larger stability constant K) in the solution occurred around the equivalence point.

Various modifications and alterations to the described embodiments will be apparent to those skilled in the art in view of the teachings herein. Accordingly, the disclosure herein is intended to be illustrative, but not limiting, of the scope of the disclosed subject matter. Moreover, the principles of the disclosed subject matter can be implemented in various configurations and are not intended to be limited in any way to the specific embodiments presented herein.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

Various patents and patent applications are cited herein, the contents of which are hereby incorporated by reference herein in their entireties.

What we claim is:

1. A titration-based analytical method for measuring Gold (Au) in Au-sulfite processing solutions, comprising:
a. adding a complexing agent having a predetermined concentration to the processing solution;
b. adding Au-sulfite solution to the processing solution;
c. adding a metallic salt having a predetermined concentration to the processing solutions;
d. determining the concentration of Au by measuring the endpoint of a back titration, wherein the complexing agent reacts with the complexing agent in a first reaction, and the metallic salt reacts with the remaining complexing agent in a second reaction.

2. The method of claim 1, wherein the first product of the first reaction has a larger stability constant than Au-sulfite.

3. The method of claim 1, wherein the second product of the second reaction has a less stability constant than the first product of the first reaction.

4. The method of claim 1, wherein the first reaction generates a first precipitation product, and the second reaction generates a second precipitation product.

5. The method of claim 1, wherein the processing solution is an electrodeposition solution.

6. The method of claim 1, wherein the processing solution comprises a plating metal selected from the group consisting of nickel, cobalt, iron, and combinations thereof.

7. The method of claim 1, wherein the complexing agent is selected from the group consisting of Thiourea, EDTA, Thiosulfate, nitrilotriacetic acid, iminodisuccinic acid, polyaspartic acid, S,S-ethylenediamine-N,N-disuccinic acid, methylglycinediacetic acid, L-Glutamic acid, N,N-diacetic acid, salts thereof, and combinations thereof.

8. The method of claim 1, further comprising an addition of a pH adjustment reagent to the processing solution.

9. The method of claim 1 further comprising an addition of a pH adjustment reagent to the processing solution before adding Au-sulfite solution.

10. The method of claim 8, wherein the pH adjustment reagent decreases pH values of the processing solution.

11. The method of claim 8, wherein the pH adjustment reagent is selected from the group consisting of nitric acid, hydrochloric acid, sulfuric acid, and combinations thereof.

12. The method of claim 1, wherein the metallic salt is selected from the group consisting of Ag, Cu, Fe, Al, and combination thereof.

13. The method of claim 1, wherein the back titration comprises determining the Au concentration based on a difference between an amount of endpoint volume from a blank analysis and endpoint of an analyte analysis.

14. The method of claim 1, further comprises tracking the titration using a Ag-billet electrode.
